(19) United States
(12) Patent Application Publication
(10) Pub. No.: US 2021/0315554 A1
(43) Pub. Date: Oct. 14, 2021

(54) TISSUE BIOPSY DEVICES

(71) Applicant: Covidien LP, Mansfield, MA (US)

(72) Inventors: Nikolai D. Begg, Wellesley, MA (US); Chad A. Pickering, Woburn, MA (US)

(21) Appl. No.: 16/843,819

(22) Filed: Apr. 8, 2020

(51) Int. Cl. A61B 10/02

(52) U.S. Cl. CPC A61B 10/0266; A61B 17/32053; A61B 10/0283

(57) ABSTRACT

A tissue biopsy system includes a biopsy needle having an elongated shaft with a distal tip configured to pierce and capture a sample of tissue. The elongated shaft includes a needle bore defined therein having a helical feature defined along an inner periphery thereof. Upon rotation of the biopsy needle and insertion of the biopsy needle into tissue, the helical feature of the needle bore advances tissue proximally into the needle bore for containment and retention.

### BACKGROUND

### Technical Field

The present disclosure relates generally to medical devices, systems, and methods. More particularly, the present disclosure relates to tissue biopsy devices and systems used in hysteroscopic surgical procedures, and methods of hysteroscopic tissue biopsy.

### Background of Related Art

Tissue biopsy is a medical procedure used to obtain a tissue sample from an area of the body. The obtained tissue sample is usually tested to assist in diagnosing a medical condition or to assess the effectiveness of a particular treatment. Endometrial biopsies are procedures employed for evaluating uterine tissue for the presence of cancerous or pre-cancerous cells. Endometrial biopsies typically include the insertion of a catheter through the cervix and into the uterus of the patient. Following insertion of the catheter, a biopsy needle is inserted into the uterus via the catheter, whereupon a small amount of endometrial lining is aspirated with the biopsy needle.

### SUMMARY

As used herein, the term “distal” refers to the portion that is described which is further from a user, while the term “proximal” refers to the portion that is being described which is closer to a user. The terms “substantially” and “approximately,” as utilized herein, account for industry-accepted material, manufacturing, measurement, use, and/or environmental tolerances. Further, any or all of the aspects and features described herein, to the extent consistent, may be used in conjunction with any or all of the other aspects and features described herein.

Provided in accordance with aspects of the present disclosure is a tissue biopsy system which includes a biopsy needle having an elongated shaft with a distal tip configured to pierce and capture a sample of tissue. The elongated shaft includes a needle bore defined therein having a helical feature defined along an inner periphery thereof. Upon rotation of the biopsy needle and insertion of the biopsy needle into tissue, the helical feature of the needle bore advances tissue proximally into the needle bore for containment and retention.

In aspects according to the present disclosure, the distal tip of the elongated shaft includes a sharpened edge. In other aspects according to the present disclosure, the outer periphery of the elongated shaft is smooth to facilitate insertion thereof.

In aspects according to the present disclosure, the needle bore is configured to rotate independently of the biopsy needle. In other aspects according to the present disclosure, the needle bore is adapted to connect to a suction source to facilitate capture and retainment of tissue.

Provided in accordance with aspects of the present disclosure is a tissue biopsy system which includes a biopsy needle having an elongated shaft with a distal tip configured to pierce and capture a sample of tissue. The elongated shaft includes a needle bore defined therein having a series of laser cut-outs defined along an inner periphery thereof. Upon rotation of the biopsy needle and insertion of the biopsy needle into tissue, the series of laser cut-outs of the needle bore advance tissue proximally into the needle bore for containment and retention.

In aspects according to the present disclosure, the distal tip of the elongated shaft includes a sharpened edge. In other aspects according to the present disclosure, the outer periphery of the elongated shaft is smooth to facilitate insertion thereof.

In aspects according to the present disclosure, the needle bore is configured to rotate independently of the biopsy needle. In other aspects according to the present disclosure, the needle bore is adapted to connect to a suction source to facilitate capture and retainment of tissue.

As used herein, the term distal refers to that portion of the device which is farthest from the user, while the term proximal refers to that portion of the device which is closest to the user. Further, to the extent consistent, any of the aspects detailed herein may be utilized with any or all of the other aspects detailed herein.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a cross-sectional view illustrating an exemplary embodiment of a tissue biopsy system including an elongate guide member inserted hysteroscopically into the uterus and engaged with uterine tissue and a biopsy needle disposed within the elongate guide member;

FIG. 2 is a side view illustrating the biopsy needle shown in FIG. 1;

FIG. 3 is a side view illustrating a distal tip of the biopsy needle shown in FIG. 2 extending through a helical distal tip of the elongate guide member shown in FIG. 1;

FIG. 4 is a side view illustrating another embodiment of a tissue biopsy system including a biopsy needle extending over a helical distal tip of an elongate guide member;

FIG. 5 is an internal top view of a tissue biopsy device according to another embodiment of the present disclosure including an interior helical retention feature; and

FIG. 6 is an internal top view of a tissue biopsy device according to another embodiment of the present disclosure including an interior helical retention feature.

### DETAILED DESCRIPTION

Embodiments of the present disclosure will now be described in detail with reference to the drawings, in which like reference numerals designate identical or corresponding elements in each of the several views. In the following description, well-known functions or constructions are not described in detail to avoid obscuring the present disclosure in unnecessary detail.

The devices, systems, and methods of the present disclosure may be used for retrieving tissue during any minimally invasive procedure. That is, although the systems and methods of the present disclosure are described below with reference to a hysteroscopic biopsy procedure, the systems and methods of the present disclosure may also be used for other minimally invasive tissue-retrieving procedures.

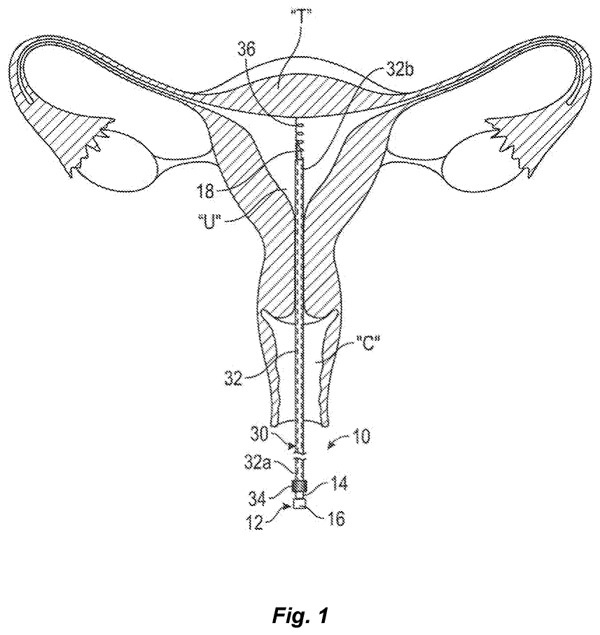

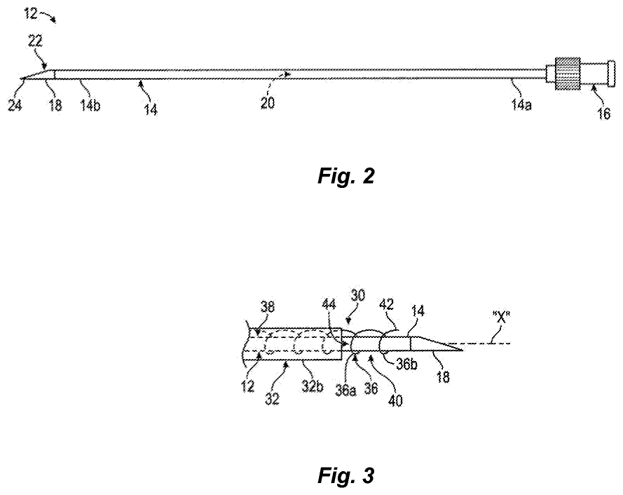

With reference to FIGS. 1-3, a tissue biopsy system 10 is configured for insertion into a tissue opening, for example, a cervix “C,” and to take a sample of tissue, for example, uterine tissue “T,” for biopsy. The tissue biopsy system 10 generally includes a biopsy needle 12 and an elongate guide member 30 for guiding the biopsy needle 12 to a target tissue site.

The biopsy needle 12 has an elongate body portion 14, a handle portion 16 coupled to a proximal end portion 14a of the elongate body portion 14, and a distal tip 18 coupled to a distal end portion 14b of the elongate body portion 14. The elongate body portion 14 may be a catheter, a cannula, a tube, or the like, and defines a longitudinally-extending passageway 20. The elongate body portion 14 may be fabricated from any suitable material including a metal or plastic, such as, for example, silicone rubber, polyurethane, PET, thermoplastic polymers, and/or nylon. The handle portion 16 is configured to be grasped by a clinician to manipulate the biopsy needle 12 to a selected position within a surgical site. In some aspects, the handle portion 14 of the biopsy needle 12 may be configured to be attached to a robotic arm assembly (not shown) for controlling movement of the biopsy needle 12.

The distal tip 18 of the biopsy needle 12 extends distally from the distal end portion 14b of the elongate body portion 14. In aspects, the distal tip 18 may be monolithically formed with the distal end portion 14b of the elongate body portion 14 or be connected thereto in any other suitable manner, e.g., via mechanical engagement, welding, adhesion, etc. The distal tip 18 is configured to pierce tissue and capture a sample of the tissue therein. The distal tip 18 may be fabricated from metal (e.g., stainless steel) and defines a hollow interior 22 configured for receipt of tissue. A distal-most end 24 of the distal tip 18 may have a lancet point configuration. It is contemplated that the distal-most end 24 of the distal tip 18 may be any suitable needle tip type of any suitable geometry and any suitable gauge (e.g., 18 gauge) to facilitate piercing tissue.

With reference to FIGS. 1 and 3, the elongate guide member 30 of the tissue biopsy system 10 includes a shaft 32, a handle portion 34 (FIG. 1), and a distal tip 36. The shaft 32 is hollow, and therefore defines a longitudinally-extending channel 38 configured for passage of the elongate body portion 14 and the distal tip 18 of the biopsy needle 12 therethrough. In some aspects, the shaft 32 may be configured as a flexible (and, in embodiments, resilient) wire. In other aspects, the shaft 32 may be a rigid linear wire or a wire having a rigid or biased helical configuration along at least a portion of its length. The shaft 32 has a proximal end portion 32a and a distal end portion 32b and defines a longitudinal axis “X” (FIG. 3).

The handle portion 34 of the elongate guide member 30 is coupled to the proximal end portion 32a of the shaft 32 and is configured to be grasped by a clinician to manipulate the elongate guide member 30 to the target tissue site. In some aspects, the handle portion 34 of the elongate guide member 30 may be configured to be attached to the robotic arm assembly for controlling movement of the elongate guide member 30.

The distal tip 36 of the elongate guide member 30 extends distally from the distal end portion 32b of the shaft 32. In aspects, the distal tip 36 of the elongate guide member 30 may be monolithically formed with or otherwise connected to the distal end portion 32b of the shaft 32, e.g., via welding, mechanical engagement, etc. The distal tip 36 of the elongate guide member 30 is an open coil helical wire, such that adjacent coils 36a, 36b of the distal tip 36 have a space 40 defined therebetween to allow for tissue to be disposed therebetween. The distal tip 36 may be configured to resist compression or may be configured to compress under a threshold force to narrow the distance between the adjacent coils 36a, 36b. It is contemplated that the distal tip 36 may have any suitable length including any suitable number of coils and pitch of coils to make up the helical configuration thereof. A further-most distal end 42 of the distal tip 36 may be sharp, pointed, or otherwise tapered, such that the distal tip 36 is configured to pierce tissue during a rotation of the shaft 32 about the longitudinal axis “X.”

The distal tip 36 of the elongate guide member 30 is also configured to guide the distal tip 18 of the biopsy needle 12 towards target tissue. In particular, the distal tip 36 of the elongate guide member 30 defines a longitudinally-extending channel 44 that is coextensive with the channel 38 of the shaft 32. As such, as the distal tip 18 of the biopsy needle 12 passes distally out of the shaft 32 of the elongate guide member 30, the distal tip 18 of the biopsy needle 12 enters the channel 44 of the distal tip 36 of the elongate guide member 30. The channel 44 of the distal tip 36 of the elongate guide member 30 has a diameter that is greater than a diameter of the distal tip 18 of the biopsy needle 12 to allow for the distal tip 18 of the biopsy needle 12 to pass therethrough during use.

In aspects, the distal tip 36 of the elongate guide member 30 may be radiopaque so that it can be seen with imaging systems such as X-ray, cone beam CT, CAT, fluoroscopy, etc. The distal tip 36 of the elongate guide member 30 may have fixation elements (e.g., barbs, teeth, hooks, or the like) disposed at a suitable distance proximal from the further-most end 42 thereof. The fixation elements (not shown) may assist in fixing the distal tip 36 in tissue and/or may provide tactile feedback to the clinician indicating that the distal tip 36 has reached a sufficient depth in tissue. In aspects, the distal tip 36 of the elongate guide member 30 may be coated with or fabricated from polytetrafluoroethene (PTFE), graphite, or other lubricating agents to minimize friction with tissue. In aspects, the distal tip 36 may be fabricated from a shape memory material (polymer or alloy), e.g., nickel titanium, such that the distal tip 36 may be configured to move from a first state, in which the distal tip 36 is linear, to a second state, in which the distal tip 36 assumes its helical configuration upon receiving an electrical impulse or upon changing to a particular temperature (e.g., body temperature).

Referring again to FIGS. 1-3, in use, the tissue biopsy system 10 may be used to sample tissue for biopsy. For example, the tissue biopsy system 10 may be utilized in performing a hysteroscopic tissue biopsy procedure. The elongate guide member 30 is positioned through a cervix “C” or other suitable natural or artificial tissue opening and guided to the target tissue site within the uterus “U” using medical imaging, such as, for example, a hysteroscope (not shown). In such embodiments, the elongated guide member 30 may be passed through a working channel of the hysteroscope or other access-providing device. Once elongate guide member 30 reaches the target tissue site, the further-most end 42 of the distal tip 36 of the elongate guide member 30 engages tissue “T” at the target tissue site within the uterus “U” and is rotated about its longitudinal axis “X.” Due to the helical configuration of the distal tip 36 of the elongate guide member 30 and the tapered configuration of the further-most end 42 thereof, the rotation of the elongate guide member 30 drives the further-most end 42 of the distal tip 36 into the tissue in a helical path. Continued rotation of the elongate guide member 30 screws the distal tip 36 into the tissue to a selected depth to fix the distal tip 36 in the tissue at the target tissue site. The selected depth may be determined by the surgeon, e.g., through visualization, or may be the maximum depth set by the elongated guide member 30, e.g., according to the length of the distal tip 36. To this end, a kit of elongate guide members 30 having different distal tip 36 lengths and/or an elongate guide member 30 having an adjustable-length distal tip 36, e.g., via telescoping the distal tip 36 relative to shaft 32 or selecting a distal tip 36 of desired length from a lot of different-length distal tips 36 and releasably engaging that distal tip 36 with shaft 32, may be provided.

With the distal tip 36 of the elongate guide member 30 fixed to the tissue, the biopsy needle 12 is positioned into the channel 38 of the shaft 32 of the elongate guide member 30 (e.g., via an entry opening at a proximal end of the shaft 32 or handle portion 34) and moved distally therethrough. The distal tip 18 of the biopsy needle 12 moves through the channel 38 of the shaft 32 and thereafter into the channel 44 of the distal tip 36. The distal tip 36 of the biopsy needle 12 is driven distally through and relative to the distal tip 36 of the elongate guide member 30 to pierce the tissue to capture a sample of the tissue in the hollow interior 22 of the distal tip 18 of the biopsy needle 12. Channel 44 guides the distal tip 18 of the biopsy needle 12 through tissue and may also serve as a visual indicator for the depth of insertion the distal tip 18 of the biopsy needle 12. That is, distal 18 may piece the tissue to a selected depth corresponding or relative to the distal-most end of distal tip 36, thus allowing surgeon to control the depth.

Upon capturing the tissue sample, the biopsy needle 12 is withdrawn proximally from the elongate guide member 30 and the elongate guide member 30 is reverse-rotated to detach the distal tip 36 thereof from the tissue to enable removal of the elongate guide member 30.

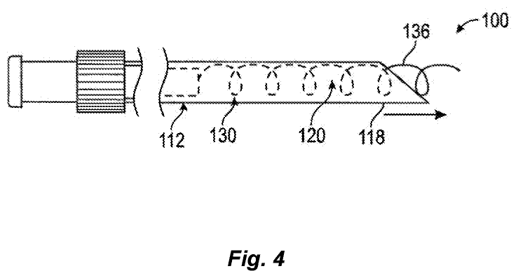

FIG. 4 illustrates another embodiment of a tissue biopsy system 100, similar to the tissue biopsy system 10 of FIGS. 1-3. The tissue biopsy system 100 includes a biopsy needle 112 and an elongate guide member 130, each similar to the biopsy needle 12 and elongate guide member 30 described above with reference to FIGS. 1-3, except as explicitly contradicted below. Therefore, the biopsy needle 112 and elongate guide member 130 are only be described in the detail necessary to elucidate distinctions from the embodiment of FIGS. 1-3.

The biopsy needle 112 defines a longitudinally-extending passageway 120 configured for receipt of a distal tip 136 of the elongate guide member 130. As such, instead of the elongate guide member 130 configured to guide the biopsy needle 112 through it, the biopsy needle 112 is slid distally over the distal tip 136 of the elongate guide member 130 while the distal tip 136 of the elongate guide member 130 remains disposed within the passageway 120 of the biopsy needle 112, thus guiding the biopsy needle 112 about the elongated guide member 130. During use, after capturing tissue within the distal tip 118 of the biopsy needle 112, the elongate guide member 130 is rotated to detach the distal tip 136 thereof from the tissue prior to withdrawing the biopsy needle 112. Alternatively, elongate guide member 130 and biopsy needle 112 may be withdrawn together with one another (with both rotating and translating or with both translating and just elongated guide member 130 rotating).

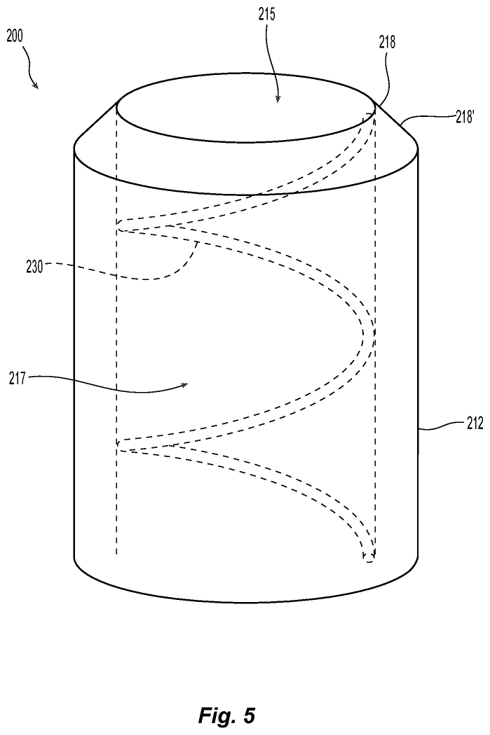

FIG. 5 illustrates another embodiment of a tissue biopsy system 200, similar to the tissue biopsy system 10 of FIGS. 1-3. The tissue biopsy system 200 includes a biopsy needle 212 having a smooth outer surface and a distal tip 218 that defines an aperture 215 configured to receive tissue core samples therethrough for retention within a needle bore 217 defined therein. In contrast to the aforedescribed embodiments in FIGS. 1-3, distal tip 218 includes a sharpened edge 218′ configured to cut tissue as the needle 212 is rotated and advanced into tissue. An interior helical feature 230 is defined within the needle bore 217 and is configured to advance the tissue core samples proximally as the needle 212 rotates.

In use, as the tubular needle 212 is rotated and advanced into tissue, the sharpened edge 218′ cuts tissue to create a cylindrical tissue core sample. The internal helical feature 230 defined within the needle bore 217 encourages the tissue core sample to advance proximally into the needle bore 217 as the needle 212 rotates and remain retained therein until the tissue core sample is removed. Following collection of the tissue core sample and removal of the needle 212 from the operating cavity, the tissue core sample may be removed from the needle bore 217 by pressure, a mechanical plunger or the like or by other known methods.

In embodiments, the depth of penetration of the needle 212 and, thus, the size of the tissue core sample, may be regulated by a variety of known mechanical features. In embodiments, the tissue core sample may be removed from the needle bore 217 by simply stabilizing the tissue core sample, e.g., holding it with tweezers, and reversing the rotation of the needle 212 wherein the helical feature 230 advances the tissue distally out of opening 215. In embodiments, the helical feature 230 may be separate from the needle 212 and independently rotated to advance the tissue core sample into the needle bore 217, e.g., on a separate inner rotating tube (Not shown). Independent rotation of the helical feature 230 may be separately controlled to adjust the speed thereof, e.g., faster, same or slower than the needle 212. In embodiments, the needle bore 317 may be adapted to connect to a suction source to facilitate capture and retainment of the tissue core sample.

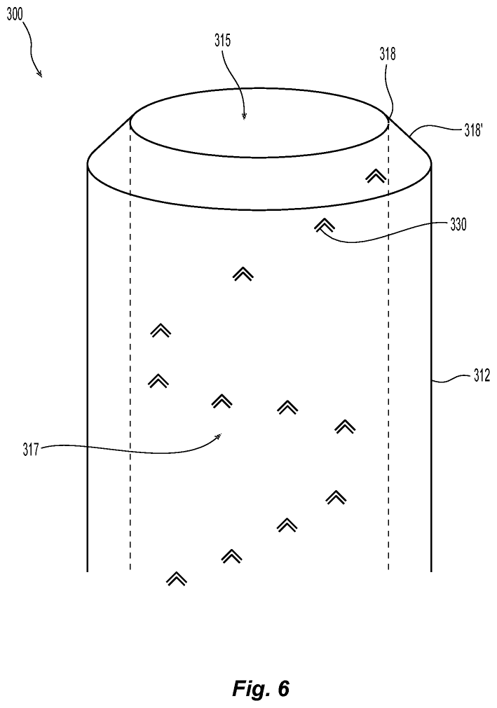

FIG. 6 illustrates another embodiment of a tissue biopsy system 300, similar to the tissue biopsy system 10 of FIGS. 1-3. The tissue biopsy system 200 includes a biopsy needle 312 having a smooth outer surface and a distal tip 318 that defines an aperture 315 configured to receive tissue core samples therethrough for retention within a needle bore 317 defined therein. In contrast to the aforedescribed embodiments in FIGS. 1-3, distal tip 318 includes a sharpened edge 318′ configured to cut tissue as the needle 312 is rotated and advanced into tissue. A series of laser cut-outs or punched-out areas 330 are defined within the needle bore 317 and are configured to advance the tissue core samples proximally as the needle 312 rotates. The series of laser cut-outs or punched-out areas 330 may be configured in a helical or similar pattern within the needle bore 317 to facilitate advancement of the tissue core sample.

In use, as the tubular needle 312 is rotated and advanced into tissue, the sharpened edge 318′ cuts tissue to create a cylindrical tissue core sample. The pattern of the series of laser cut-outs or punched-out areas 330 defined within the needle bore 317 encourage the tissue core sample to advance proximally into the needle bore 317 as the needle 312 rotates and remain retained therein until the tissue core sample is removed.

Following collection of the tissue core sample and removal of the needle 312 from the operating cavity, the tissue core sample may be removed from the needle bore 317 by pressure, a mechanical plunger or the like or by other known methods.

In embodiments, the depth of penetration of the needle 312 and, thus, the size of the tissue core sample, may be regulated by a variety of known mechanical features. In embodiments, the tissue core sample may be removed from the needle bore 317 by simply stabilizing the tissue core sample, e.g., holding it with tweezers, and reversing the rotation of the needle 312 wherein the pattern of the laser cut-outs or punched-out areas 330 advances the tissue distally out of opening 315.

In embodiments, the pattern of the laser cut-outs or punched-out areas 330 may be separate from the needle 312 and independently rotated to advance the tissue core sample into the needle bore 317, e.g., on a separate inner rotating tube (Not shown). Independent rotation of the pattern of the laser cut-outs or punched-out areas 330 may be separately controlled to adjust the speed thereof, e.g., faster, same or slower than the needle 312. In embodiments, the needle bore 317 may be adapted to connect to a suction source to facilitate capture and retainment of the tissue core sample.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A tissue biopsy system, comprising:
a biopsy needle having an elongated shaft with a distal tip configured to pierce and capture a sample of tissue, the elongated shaft including a needle bore defined therein having a helical feature defined along an inner periphery thereof, wherein upon rotation of the biopsy needle and insertion of the biopsy needle into tissue, the helical feature of the needle bore retracts tissue proximally into the needle bore for containment and retention.

2. The tissue biopsy system according to claim 1, wherein the distal tip of the elongated shaft includes a sharpened edge.

3. The tissue biopsy system according to claim 1, wherein the outer periphery of the elongated shaft is smooth to facilitate insertion thereof.

4. The tissue biopsy system according to claim 1, wherein the needle bore is a concentric tube configured to rotate independently of the biopsy needle.

5. The tissue biopsy system according to claim 1, wherein the needle bore is adapted to connect to a suction source to facilitate capture and retention of tissue.

6. A tissue biopsy system, comprising:
a biopsy needle having an elongated shaft with a distal tip configured to pierce and capture a sample of tissue, the elongated shaft including a needle bore defined therein having a series of laser cut-outs defined along an inner periphery thereof, wherein upon rotation of the biopsy needle and insertion of the biopsy needle into tissue, the series of laser cut-outs of the needle bore retract tissue proximally into the needle bore for containment and retention.

7. The tissue biopsy system according to claim 6, wherein the distal tip of the elongated shaft includes a sharpened edge.

8. The tissue biopsy system according to claim 6, wherein the outer periphery of the elongated shaft is smooth to facilitate insertion thereof.

9. The tissue biopsy system according to claim 6, wherein the needle bore is a concentric tube configured to rotate independently of the biopsy needle.

10. The tissue biopsy system according to claim 6, wherein the needle bore is adapted to connect to a suction source to facilitate capture and retainment of tissue.
